(19) United States
(12) Patent Application Publication
(10) Pub. No.: US 2005/0004018 A1
(43) Pub. Date: Jan. 6, 2005

(54) Use of antitumoral compound in cancer therapy

(76) Inventors: Jose Jimeno, Madrid (ES); Ana Ruiz Casado, Madrid (ES); Luis Lopez Lazaro, Madrid (ES); Eric Rowensky, San Antonio, TX (US); Manuel Hidalgo, Baltimore, MD (US)

Correspondence Address: FISH & RICHARDSON PC, 225 FRANKLIN ST, BOSTON, MA, 02110, US

(21) Appl. No.: 10/492,320

(22) PCT Filed: Oct. 21, 2002
(86) PCT No.: PCT/US02/33548

Related U.S. Application Data
(60) Provisional application No. 60/348,414, filed on Oct. 19, 2001.

(51) Int. Cl. A61K 38/17

(52) U.S. Cl. 514/12

(57) ABSTRACT

Improved dosing schedules for ecteinascidin 743 are given for treatment of cancer.

### FIELD OF THE INVENTION

The present invention is directed to the use of ecteinascidin 743 and products containing this compound for cancer therapy, in particular to improvements in the use of ecteinascidin 743 in the treatment of cancer.

### BACKGROUND OF THE INVENTION

Cancer comprises a group of malignant neoplasms that can be divided into two categories, carcinoma, comprising a majority of the cases observed in the clinics, and other less frequent cancers, which include leukemia, lymphoma, central nervous system tumours and sarcoma. Carcinomas have their origin in epithelial tissues while sarcomas develop from connective tissues and those structures that had their origin in mesoderm tissues. Sarcomas can affect, for instance, muscle or bone and occur in the bones, bladder, kidneys, liver, lung, parotid, spleen, etc.

Cancer is invasive and tends to metastasise to new sites. It spreads directly into surrounding tissues and also may be disseminated through the lymphatic and circulatory systems.

Many treatments are available for cancer, including surgery and radiation for localised disease, and drugs. However, the efficacy of available treatments on many cancer types is limited, and new, improved forms of treatment showing clinical benefit are needed. This is especially true for those patients presenting with advanced and/or metastatic disease. It is also true for patients relapsing with progressive disease after having been previously treated with established therapies for which further treatment with the same therapy is mostly ineffective due to acquisition of resistance or to limitations in administration of the therapies due to associated toxicities.

Chemotherapy plays a significant part in cancer treatment, as it is required for treatment of advanced cancers with distant metastasis and often helpful for tumor reduction before surgery, and many anti-cancer drugs have been developed based on various modes of action.

The ecteinascidins (herein abbreviated Et or Et's) are exceedingly potent antitumor agents isolated from the marine tunicate Ecteinascidia turbinata. Several ecteinascidins have been reported previously in the patent and scientific literature. See, for example U.S. Pat. No. 5,256,663, which describes pharmaceutical compositions comprising matter extracted from the tropical marine invertebrate, Ecteinascidia turbinata, and designated therein as ecteinascidins, and the use of such compositions as antibacterial, anti-viral, and/or antitumor agents in mammals; U.S. Pat. No. 5,089,273, which describes novel compositions of matter extracted from the tropical marine invertebrate, Ecteinascidia turbinata, and designated therein as ecteinascidins 729, 743, 745, 759A, 759B and 770. These compounds are useful as antibacterial and/or anti-tumor agents in mammals; U.S. Pat. No. 5,478,932, which describes ecteinascidins isolated from the Caribbean tunicate Ecteinascidia turbinata, which provide in vivo protection against P388 lymphoma, B16 melanoma, M5076 ovarian sarcoma, Lewis lung carcinoma, and the LX-1 human lung and MX-1 human mammary carcinoma xenografts.

One of them, ecteinascidin-743 (ET-743), is a novel tetrahydroisoquinoline alkaloid isolated from the marine ascidian Ecteinascidia turbinata that has considerable antitumor activity in murine and human tumors in vitro.

In a study of human cancer cell lines, ET-743 exhibited extremely potent activity against several soft tissue sarcoma cell lines with IC₅₀s well below 1 pM. See for example Li W, Jhanwar S, Elisseyeff Y, Bertino J R. “Potent antitumor activity of ET-743 against human soft tissue sarcoma cell lines”, Clin Cancer Res 1999; 5: 305 and Izbicka E, Lawrence R, Raymond E, et al.: “In vitro antitumor activity of the novel marine agent, Ecteinascidin-743 against human tumors explanted from patients”, Ann. Oncol. 1998; 9: 981-7.

Potent antitumor activity has been demonstrated in a broad range of in vivo tumor models, including human tumor xenografts in nude mice. This is illustrated in Valoti G, Nicoletti M I, Faircloth G, et al.: “Antitumor effect of ecteinascidin-743 (ET-743) on human ovarian carcinoma xenografts”, Proc. Am. Assoc. Cancer Res. 1997; 38: 1477; Faircloth G, Hendriks H R, Giavazzi R, et al.: “In vivo antitumor activity of Ecteinascidin 743 (ET 743), a novel marine derived cytotoxic against human xenografts tumor models”, Ann Oncol 1996; 7: 125; Hendriks H R, Fiebig H H, Giavazzi R, et al.: “High antitumour activity of ET743 against human tumour xenografts from melanoma, non-small-cell lung and ovarian cancer” Ann. Oncol. 1999; 10: 1233-40.

Et-743 has a novel complex mechanism of action at the level of gene transcription. ET-743 binds to guanine-cytosine rich sequences in the minor groove of DNA and alkylates guanine residues at the N2 position, see Pommier Y, Kohlhagen G, Ballly C, et al.: “DNA sequence- and structure-selective alkylation of guanine N2 in the DNA minor groove by Ecteinascidin 743, a potent antitumor compound from the Caribbean tunicate Ecteinascidia turbinata”, Biochemistry 1996; 35: 13303-9. Cell cycle studies have demonstrated that ET-743 decreases the rate of progression of tumor cells through S-phase and causes prolonged p53-independent blockade in G₂/M, giving rise to a strong apoptotic response, Erba W. Bergamaschi D, Ronzoni S, et al.: “Mode of action of Ecteinascidin 743, a natural marine compound with antitumor activity” Ann. Oncol. 1998; 9: 535. Cells in G₁ are more sensitive to the cytotoxic effects of ET-743 than cells in S-phase or G2/M. These effects appear to be mediated by multiple mechanisms. ET-743 strongly inhibits the activation of the transcription of certain genes, including p21, c-fos, c-jun and mdr1, without affecting their basal transcription levels. Further background concerning this point is to be found in Mantovani R, La Valle E, Bonfanti M, et al.: “Effect of ET-743 on the interaction between transcription factors and DNA”, Ann. Oncol. 1998; 9: 534; Minuzzo M, Marchini S, Broggini M, et al.: “Interference of transcriptional activation by the antineoplastic drug ecteinascidin-743”, Proc. Natl. Acad. Sci. USA 2000; 97: 6780-4; Jin S, Gorfajn B, Faircloth G, Scotto K W.: “Ecteinascidin 743, a transcription-targeted chemotherapeutic that inhibits MDR1 activation”, Proc. Natl. Acad. Sci. USA 2000; 97: 6775-9; Synold T W, Dussault I, Forman B M.: “The orphan nuclear receptor SXR coordinately regulates drug metabolism and efflux”, Nat. Med. 2001; 7: 584-90.

Toxicological evaluations of ET-743 as a single or a fractionated dose by the intravenous route in mice, rats, and dogs have consistently shown the potential of ET-743 to induce reversible hematological and hepatic toxicity. Liver toxicity was evident from transient increase in serum levels of liver enzymnes, bilirubin and bile acids, and from histopathological changes in the liver. Further toxicity included lesions at the site of injection, spleen and thymus lesions, bile duct hyperplasia, portal fibrosis, gall bladder lesions characterized by cholecystitis with oedema and a lymphocytic infiltrate, pancreatic acinar cell atrophy and apoptosis, and decreased testicular and ovarian weights. Studies in dogs showed vomiting and diarrhoea following the administration of ET-743. A study in cynomolgus monkeys confirmed the potential of single doses of ET-743 to induce hepatic and hematological toxicity, emesis and diarrhoea. However, fractionated dosing induced only minor toxicity in monkeys. See Jimeno J, Faircloth G, Cameron L, et al.: “Progress in the acquisition of new marine.derived anticancer compounds: development of ecteinascidin-743 (ET-743)”, Drugs Future 1996; 21: 1155-65.

An in vitro bone marrow assay using human, murine and canine progenitor cells, showed equal sensitivity of erythropoid and myeloid cells to ET-743. Prolonged or repeated exposure to the drug proved more toxic to hematopoietic progenitors than a single 1-hour exposure, see for example Ghielmini M, Colli E, Erba E, et al.: “In vitro schedule-dependency of myelotoxicity and cytotoxicity of Ecteinascidin 743 (ET-743)”, Ann. Oncol. 1998; 9: 989-93. The therapeutic index of ET-743 was more favourable with prolonged exposure.

A clinical development program of ET-743 in cancer patients was started with phase I studies investigating 1-hour, 3-hour, 24-hour and 72-hour intravenous infusion schedules and a 1 hour daily×5 (d×5) schedule. These studies are reported in Taamma A, Misset J L, Riofro M, et al.: “Phase I and pharmacokinetic study of ecteinascidin-743, a new marine compound, administered as a 24-hour continuous infusion in patients with solid tumors”, J. Clin. Oncol. 2001; 19: 1256-65; Van Kesteren C, Cvitkovic E, Taamma A, et al.: “Pharmacokinetics and pharamacodynamics of the novel marine derived anticancer agent ecteinascidin 743 in a phase I dose-finding study”, Clin. Cancer Res. 2000; 6: 4725-32; Ryan D P, Supko J G, Eder J P, et al.: “Phase I and pharmacokinetic study of ecteinascidin 743 administered as a 72-hour continuous intravenous infusion in patients with solid malignancies” Clin. Cancer Res. 2001; 7: 231-42; Villalona-Calero M A, Eckhardt S G, Weiss G, et al.: “A phase I and pharmacokinetic study of ecteinascidin-743 on a daily×5 schedule in patients with solid malignancies”, Clin. Cancer Res. 2002; 8: 75-85. Further detail on the use of Et-743 for the treatment of the human body for cancer is given in WO 0069441.

Summary results of each phase I study are detailed below:

24hr infusion every 3 weeks: A total of 52 patients were treated at nine different dose levels and received a total of 158 cycles. The MTD (maximum tolerated dose) and the RD (recommended dose) were determined at 1800 μg/m² and 1500 μg/m². The DLT (dose limiting toxicity) was hematotoxicity (i.e., neutropenia and thrombocytopenia). At the RD, transient and reversible increases in transaminases were observed in most patients. Grade 3 or 4 transaminases occurred in 68% of patients and 38% of cycles. Grade 2 or greater hyperbilirubinemia was reported in 30% of the treatment courses at the RD. Other toxicities included nausea/vomiting and asthenia.

72 hr infusion every 3 weeks: In this study, 21 adult patients were evaluated at four dose levels of ET-743. Non-hematological dose-limiting toxicity with 1200 μg/M² (MTD) was reversible grade 4 transaminases elevation (in 2 out of 9 patients), which was considered a DLT in this study. A third patient at this dose level experienced grade 4 rhabdomyolysis, grade 4 febrile neutropenia, and grade 4 thrombocytopenia. At the RD (1050 μg/M²) there was not any grade 4 hypertransaminasemia, and grade 3 was reported in 50% of patients. Increases in bilirubin and alkaline phosphatase occurred less often and were below grade 2. Nausea/vomiting and fatigue were also reported . Prolonged infusion of ET-743 over 72 hours did not allow the administration of a higher total dose per cycle than over 24 hours.

1 hr infusion days 1-5 every 3 weeks: 42 patients were treated at 10 dose levels ranging from 6 to 380 μg/m²/day. At the 380 μg/m²/day dose level (MTD), 3 patients developed DLT gong-lasting neutropenia) and one of them died because of toxicity. At the 325 μg/m²/day dose level (RD), 59% of cycles were delayed, most of them because of treatment related toxicity. Furthermore, the median day of recovery to grade 1 (at which treatment could be recommended) for neutropenia was 28 days. Thus, in some patients it may be necessary to administer the RD of 325 μg/m²/day ET-743 on a 4 week schedule. There were not any grade 4 transaminases toxicities even at the MTD. Reversible grade 3 transaminases occurred in 14% of cycles and 31% of patients at the recommended dose. An event of grade 3 hyperbilirubinemia occurred at the RD.

1 and 3 hr infusion every 3 weeks: In the first part of the study 40 patients were treated with ET-743 given as a 1-hr intravenous infusion every 3 weeks. A maximum tolerated dose (MTD) of 1100 μg/m² was defined. The dose limiting toxicities (DLT) were: grade 4 fatigue, grade 4 neutropenia lasting more than 5 days and grade 4 thrombocytopenia. At the MTD level, emesis and grade 3-4 transaminases elevation, which in some cycles did not recover by day 21, were observed too. It was realized that the MTD and recommended dose (RD) in this study were much lower than in a concurrent study with ET-743 given as a 24-hr continuous infusion. Since the latter schedule requires in-patient treatment, it was felt that a shorter infusion time was preferable if the same dose level could be reached. Therefore, the protocol was amended to assess the feasibility of ET-743 given as a 3-hr infusion. Prolonging the infusion from 1 to 3 hours resulted in a higher MTD and RD for Phase II studies (1800 and 1650 μg/M²) with a similar toxicity profile. 32 patients were treated in the second part of the study. DLTs included again G4 thrombocytopenia and G3 fatigue. Other toxicities included elevation of transaminases and increases of bilirubin G2 or greater in 9% of treatment courses at the RD. Decreases of leukocytes and neutrophil counts, asthenia, nausea/vomiting and phlebitis were also observed. Liver toxicity was evident in many patients from marked increases in serum ALT and AST and signs of cholestasis as evidenced by increases in alkaline phosphatase and bilirubin, although these occurred less frequently and were of lower grade than the increases in transaminases.

Two schedules (24 hour every 3 weeks and 3 hour every 3 weeks) reached the phase II programme. The phase II programme confirmed the activity against soft tissue sarcoma and ovarian cancer. However, the recommended starting dose for the 3 hour schedule had to be reduced because of serious toxicity. The recommended dose for the 24 hour schedule was 1500 μg /m² and the recommended dose for the 3 hour schedule is at present 1300 μg/m².

Serious increases of transaminases (grade 3-4) were more frequent with the 3 hour schedule than with the 24 hour schedule. Increased transaminases were seen in 83.4% of patients and 58.3% of cycles. This toxicity has improved with the amendment that reduces the starting dose to 1300 (grade 3 and 4 ALT in 38% and 8.5% of patients), although it would be desirable to reduce it further. Cholestasis is less severe and less frequent observing grade 1, 2 and 3 alkaline phosphatase in 50.4%, 6.5% and 1.7% (leading to an overall 58.6% that compares similarly with 57.7% over 24 hours) of patients. Grades 1-3 of bilirubin in 45% of patients was observed (versus 23.8% of patients receiving the drug over 24 hours). An infrequent but important toxicity was the renal toxicity, represented by creatinine abnormalities, that is also higher than in the 24 hour schedule.

The 3 hour every three weeks schedule has the signifcant advantage of being more confortable for the patient because it reduces the time being spent at the hospital for infusion and monitoring, in particular avoiding overnight stay. However, the schedule exhibited a greater toxicity as mentioned above and as illustrate by the following tables:

Hematological Toxicity. Worst grade per patient.
24 hour infusion

| | | NCI-CTC Grade-Number of patients (%) | | | |
|---|---|---|---|---|---|
| | N | 0-1 | 2 | 3 | 4 |
| Neutrophils | 319 | 103 (32.3) | 54 (16.9) | 92 (28.8) | 70 (21.9) |
| Platelets | 319 | 260 (81.5) | 17 (5.3) | 35 (11.0) | 7 (2.2) |
| Hemoglobin | 320 | 165 (51.6) | 111 (34.7) | 33 (10.3) | 11 (3.4) |

Liver Toxicity. Worst Grade Per Patient. 24 hour Infusion

| | | NCI-CTC Grade-Number of patients (%) | | | |
|---|---|---|---|---|---|
| | N | 1 | 2 | 3 | 4 |
| Bilirubin | 320 | 49 (15.3) | 23 (6.9) | 4 (1.3) | |
| Alk Phosph | 319 | 149 (46.7) | 29 (9.1) | 6 (1.9) | |
| Gamma GT | 109 | 25 (22.9) | 29 (26.6) | 32 (29.4) | 2 (1.8) |
| SGOT/AST | 319 | 74 (23.2) | 88 (27.6) | 120 (37.6) | 16 (5.0) |
| SGPT/ALT | 320 | 62 (19.4) | 83 (25.9) | 127 (39.7) | 30 (9.4) |

Hematological Toxicity. Worst grade per patient. 3 hour infusion

| | | NCI-CTC Grade-Number of patients (%) | | | |
|---|---|---|---|---|---|
| | N | 0-1 | 2 | 3 | 4 |
| Neutrophils | 243 | 59 (24.5) | 42 (17.4) | 57 (23.6) | 85 (35.2) |
| Platelets | 241 | 170 (69.1) | 26 (10.6) | 31 (12.6) | 14 (5.6) |
| Hemoglobin | 239 | 132 (55) | 80 (33.4) | 23 (9.6) | 4 (1.6) |

Liver toxicity. Worst grade per patient. 3 hour infusion

| | | NCI-CTC Grade-Number of patients (%) | | | |
|---|---|---|---|---|---|
| | N | 1 | 2 | 3 | 4 |
| Bilirubin | 228 | 59 (25.8) | 38 (16.6) | 6 (2.6) | 0 |
| Alk Phosph | 228 | 115 (50.4) | 15 (6.5) | 4 (1.7) | 2 (0.8) |
| SGOT/AST | 232 | 17 (7.2) | 29 (12.3) | 117 (49.7) | 58 (24.6) |
| SGPT/ALT | 233 | 13 (5.5) | 19 (8.1) | 107 (45.7) | 87 (37.1) |

Creatinine abnormalities. 3 hour infusion

| | | NCI-CTC Grade | | | |
|---|---|---|---|---|---|
| | N | 1 | 2 | 3 | 4 |
| Per patient | 218 | 76 (23.9) | 19 (6.0) | 3 (0.9) | 2 (0.6) |
| (24 h) | | | | | |
| Per patient | 218 | 44 (20.1) | 10 (4.6) | 5 (2.3) | 0 |
| (3 h) | | | | | |

It is an object of the present invention to provide a cancer therapy using Et-743 which allows for short infusion times while minimizing toxicities induced by the administration of ET-743, and without sacrificing the desired antineoplastic effects.

### SUMMARY OF THE INVENTION

We have now found, unexpectedly, that a different type of schedule and dosage allows for an effective cancer therapy with ET-743. Surprisingly, our results show that it is possible to administer ET-743 with reduced infusion times while avoiding toxicities and mantaining the desired antineoplastic effects. It is significant that the most frequent toxicities have been reduced more than 3 times, transaminases toxicity is reduced up to 8 times less than the 3 hour every three weeks schedule and serious renal toxicity is avoided.

The present invention provides a method of treating cancer in humans, comprising intravenously infusing a composition comprising ET-743 into a human having cancer at continuous dosage over a period up to 4 hours, wherein the step of infusing is repeated weekly on a cyclic basis.

The infusing step is typically repeated on a cyclic basis. The cyclic basis comprises two phases, the phase of weekly infusing and a phase of not infusing, referred to a rest phase. In the rest phase the patients are allowed to recover. Usually the cycle is worked out in weeks, and thus the cycle comprises one or more weeks of an infusion phase, and one or more weeks of a rest phase. Preferably the rest period is not longer than the infusion phase. Thus, preferably the rest phase is the same number of weeks as the infusion phase, or a lesser number of weeks. Particularly preferred is for the infusion phase to be a greater number of weeks than the rest phase, though a cycle of one week infusion and one week rest is envisaged. Preferably the resting phase is one week within each cycle. The preferred duration of each cycle is of 2 to 4 weeks; multiple cycles can be given as needed. A cycle of 4 weeks is most preferred.

In a particular embodiment, the infusion time is between 1 and 3 hours, preferably between 2 and 3 hours. Especially preferred is a time of about 3 hours.

In another embodiment of the invention, the dosage of Et-743 is below 650 μg/m²/weekly, preferably between 300 and 600 μg/m²/weekly, more preferably between 400 and 600 μg/m²/weekly. Suitably the dosage is between 525 and 600 μg/m²/weekly, especially preferred is a dosage of about 580 μg/m²/weekly.

The above schedules and dosages allow for an effective cancer therapy in humans, while avoiding toxicities. This means that with these dosages and schedules the therapuetic index is improved. We have found that ET-743 is effective in the treatment of several cancer types, including advanced or metastatic. Preferably, ET-743 is used according to the above schedules and dosages for the treatment of sarcoma, osteosarcoma, ovarian cancer, breast cancer, melanoma, colorectal cancer, mesothelioma, renal cancer, endometrial cancer and lung cancer.

The present invention also provides a pharmaceutical composition containing a recommended dose of ET-743 for weekly administration and a pharmaceutically acceptable carrier.

In a further aspect of the present invention, a medical kit for administering ET-743 is provided, comprising printed instructions for administering ET-743 according to the dosing schedules set forth above, and a supply of ET-743 in dosage units for at least one cycle, wherein each dosage unit contains the appropriate amount of ET-743 for the treatments as defined above and a pharmaceutically acceptable carrier.

### DETAILED DESCRIPTION

ET-743 is a natural compound represented by the following formula:

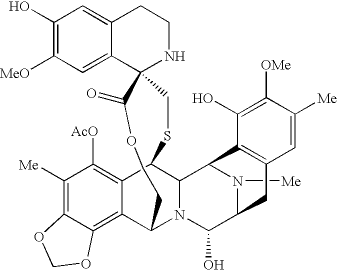

ET-743 is supplied and stored as a sterile lyophilized product, consisting of ET 743 and excipient in a formulation adequate for therapeutic use, in particular a formulation containing mannitol and a phosphate salt buffered to an adequate pH.

A preferred formulation, which shows improved stability at higher storage temperature, is one which after dilution contains per ml. 0.05mg of ET743, 50 mg of mannitol and 6.8 mg of potassium dihydrogen phosphate to adjust to a pH between 4.00 and 6.00, with 4.80 being the preferred pH. The product is lyophilized and stored in the cold, between +4 C and −20 C and protected from light until use.

Preparation of the reconstituted solution is performed under aseptic conditions by adding distilled water in the amount of 5 ml for every 0.25 mg of ET-743 and shaking for a short time to dissolve the solids.

Preparation of the infusion solution is also performed under aseptic conditions by withdrawing the reconstituted solution volume corresponding to dosage calculated for each patient, and slowly injecting the required reconstituted solution volume into an infusion bag or bottle containing between 100 and 1000 ml of 0.9% sodium chloride solution, after which the whole is homogenised by slow manual shaking. The ET-743 infusion solution should be administered intravenously, as soon as possible, within 48 hours after preparation. PVC and polyethylene infusion systems, as well as clear glass are preferred container and conduit materials.

The administration is performed in cycles, in the application method of the invention, an intravenous infusion of ET743 is given to the patients every week, allowing for a resting phase in each cycle in which the patients recover. Preferably the resting phase is one week within each cycle. The preferred duration of each cycle is of 2 to 4 weeks; multiple cycles can be given as needed. Dose delays and/or dose reductions and schedule adjustments are performed as needed depending on individual patient tolerance of treatments, in particular does reductions are recommended for patients with higher than normal serum levels of liver transaminases or alkaline phosphatase, or bilirubin.

Depending on the type of tumour and the developmental stage of the disease, the treaments of the invention are useful in preventing the risk of developing tumours, in promoting tumour regression, in stopping tumour growth and/or in preventing metastasis.

Although guidance for the dosage is given above, the correct dosage of the compound will vary according to the particular formulation, the mode of application, and the particular situs, host and tumour being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

The Recommended Dose (RD) is the highest dose which can be safely administered to a patient producing tolerable, manageable and reversible toxicity according to the Common Toxicity Criteria (CTC) established for example by the National Cancer Institute, (USA) typically with no more than 2 out of 6 patients presenting any dose limiting toxicities (DLT). Guidelines for cancer therapy frequently call for administration of chemotherapeutic agents at the highest safe dose at which toxicity is manageable in order to achieve maximum efficacy (DeVita, V. T. Jr., Hellman, S. and Rosenberg, S. A., Cancer: Principles and Practice of Oncology, 3rd ed., 1989, Lipincott, Philadelphia). For ET-743, the recommended doses are as defined above and set forth in the examples.

The compound ET743 and compositions of this invention may be used with other drugs to provide a combination therapy. The other drugs may form part of the same composition, or be provided as a separate composition for administration at the same time or a different time. The identity of the other drug is not particularly limited, and suitable candidates include:
a) drugs with antimitotic effects, especially those which target cytoskeletal elements, including microtubule modulators such as taxane drugs (such as taxol, paclitaxel, taxotere, docetaxel), podophylotoxins or vinca alkaloids (vincristine, vinblastine);
b) antimetabolite drugs (such as 5-fluorouracil, cytarabine, gemcitabine, purine analogues such as pentostatin, methotrexate);
c) alkylating agents or nitrogen mustards (such as nitrosoureas, cyclophosphamide or ifosphamide);
d) drugs which target DNA such as the antracycline drugs adriamycin, doxorubicin, pharmorubicin or epirubicin;
e) drugs which target topoisomerases such as etoposide;
f) hormones and hormone agonists or antagonists such as estrogens, antiestrogens (tamoxifen and related compounds) and androgens, flutamide, leuprorelin, goserelin, cyprotrone or octreotide;
g) drugs which target signal transduction in tumour cells including antibody derivatives such as herceptin;
h) alkylating drugs such as platinum drugs (cis-platin, carbonplatin, oxaliplatin, paraplatin) or nitrosoureas;
i) drugs potentially affecting metastasis of tumours such as matrix metalloproteinase inhibitors;
j) gene therapy and antisense agents;
k) antibody therapeutics;
l) other bioactive compounds of marine origin, notably the didemnins such as aplidine;
m) steroid analogues, in particular dexamethasone;
n) anti-inflammatory drugs, including nonsteroidal agents (such as acetaminophen or ibuprofen) or steroids and their derivatives in particular dexamethasone; and
o) anti-emetic drugs, including 5HT-3 inhibitors (such as gramisetron or ondasetron), and steroids and their derivatives in particular dexamethasone.

Particularly preferred for use in combination therapy are dexamethasone, doxorubicin, cisplatin, paclitaxel and dexamethasone. Further guidance on combination therapy is given in WO 0236135, incorporated herein by reference in its entirety.

### EXAMPLES

A phase I clinical trial was carried out with the following protocol:

### PROTOCOL:

Dose schedule: ET-743 will be administered every week as a 3hours iv infusion for 3 consecutive weeks every 4 weeks.

Starting dose and dose escalation: The starting dose will be 300 μg/m² weekly of ET-743 given as a intravenous infusion over 3 hours for 3 consecutive weeks every 4 weeks. Patients will be sequentially enrolled into the following dose cohorts beginning at dose Level 1. A minimum cohort size of 3 patients will be treated at each of the dose levels.

ET-743 Dose Escalation Scheme

| | |
|---|---|
| Dose Level −1 | 200 μg/m² weekly |
| Dose Level 1 | 300 μg/m² weekly |
| Dose Level 2 | 400 μg/m² weekly |
| Dose Level 3 | 525 μg/m² weekly |
| Dose Level 4 | 650 μg/m² weekly |
| Dose Level 5 | 775 μg/m² weekly |
| Dose Level 6 | 900 μg/m² weekly |

Accrual at the next higher dose level: At least 1 patient at each dose level must have completed 1 cycle of therapy and two patients must have completed treatment on day 15 before a new patient can be treated at the next highest dose.
Conditions for retreatment: Patients are eligible for retreatment with intolerable toxicity, the patient desires further treatment, and fulfill the eligibility criteria.
Dose Limiting Toxicities (DLTs)

Dose limiting toxicities (DLT) will be defined as follows:
ANC <500/μL for longer than 5 days.
ANC <500/μL accompanied by fever (at least 100.5° F.).
Platelets <25,000/μL.
Any grade 3-4 nonhematological toxicity except nausea/vomiting (provided the patient is receiving an optimal antiemetic regimen consisting of dexamethasone and a serotonin antagonist on an optimal dose-schedule for prophylaxis and management), alopecia. Grade 3 or 4 elevation in trasaminases that result in either omission of 2 scheduled treatments within a cycle or delay in the initiation of a subsequent course exceeding 2 weeks.
Missing at least 2 scheduled treatments during a single course due to drug-induced toxicity (missed doses will not be made up).
Delay in the initiation of a subsequent course of treatment exceeding 2 weeks.
   Determination of the Maximum Tolerated Dose (MTD)

Cohort of 3 patients will be treated at each dose level. If no DLT is seen during the first cycle in the cohort of patients at any given dose level, new patients may be treated at the next higher level.

If any patient encounters drug-induced DLT during either cycle 1 or 2, a maximum of 6 patients may be treated at that level. If DLT is not observed in the additional patients, new patients may be treated at the next higher dose level.

If at least 2 patients experience DLT at any given dose level, this dose level will be considered the Maximum Tolerated Dose (MTD). However, it is possible that additional patients may experience DLT due to the timing of patient enrollment into that dose level.

Recommended Dose for phase II studies (RD)

Once an MTD level is established, subsequent patients should be treated at the next lower dose level. Intermediate doses may be used in some instances and flexibility is an integral part of the protocol. If two or more patients experience DLT at the lower dose level, then the MTD has again been established and additional patients will be treated at the next lower dose (unless sufficient numbers of patients have already been treated at that dose level).

The RD is defined as the highest dose level at which less than 2 of 6 patients experience DLT during cycles 1 or 2. At the RD, sufficient numbers of patients will be accrued so at least 6 patients receive at least 2 cycles of therapy, and at least 4 patients receive at least 4 courses of treatment.

### Results:

This trial was launched in May 00 and last patient was included in March 02. 31 patients were treated.

Tumours included: Sarcomas (19), UOT (1), Lung (1), Ovary (4), Breast (2), Uterus (1), Melanoma (2), Colorectal (1)

| Dose level | Patients |
|---|---|
| 300 | 3 |
| 400 | 3 |
| 525 | 4 |
| 650 | 6 |
| 580 | 15 |

2 DLT defined the MTD in this trial: Long lasting grade 3 neutropenia, and g3 Bilirubin toxicity. Both DLTs were found at the 4th level. So, the MTD in this trial was 650 mcg/sm weekly×3/4 weeks. The recommended dose is 580 mcg/sm×3 every 4 weeks.

### Toxicities

After evaluating 29 patients, grade 3-4 neutropenia was 10.3% per patient and G3 transaminases 10%.

The following tables display the main features of the toxicity seen with this schedule.

Hematologic toxicity per patient. 3 hour
weekly ×3/4 weeks infusion

| | Number | | | |
|---|---|---|---|---|
| | patients | G2(%) | G3(%) | G4(%) |
| Hemoglobin | 29 | 9(31) | 1(3.4) | 0 |
| Platelets | 29 | 1(3.4) | 0 | 0 |
| Neutrophils | 29 | 1(3.4) | 2(6.9) | 1(3.4) |

Nonhematologic toxicity per patient. 3 hour
weekly ×3/4 weeks infusion

| | Number | | | |
|---|---|---|---|---|
| | patients | G2(%) | G3(%) | G4(%) |
| Creatinine | 29 | 1(3.4) | 0 | 0 |
| Creatine | 29 | 1(3.4) | 0 | 1(3.4) |
| Kinase | | | | |
| Bilirubin | 29 | 0 | 1(3.4) | 0 |
| ALT | 29 | 9(31) | 3(10.3) | 0 |
| Alk Phos | 29 | 1(3.4) | 2(6.9) | 0 |

According to the previously depicted toxicity, this schedule shows an excellent profile of toxicity, improving the previous one (obtained with the 24 and 3 hours schedule every three weeks). It can be seen on the table that neutropenia, thrombopenia, transaminases increases and creatinine (the most frequent adverse events) have now a much lower frequency.

Bilirubin and creatinin-kinase seem to be higher than with the previous ones. 3.4% in the weekly trial means that 1 patient had this toxicity and could be unrepresentative. More patients need to be treated to confirm if this will be the true incidence.

| Grade 3-4 | 24 h | 3 h | Weekly |
|---|---|---|---|
| Hemoglobin | 13.7 | 11.3 | 3.4 |
| Platelets | 13.2 | 18.7 | 0 |
| Neutrophils | 50.7 | 58.3 | 10.3 |
| Creatinine | 0.7 | 2.3 | 0 |
| Creatinine | 2.2 | 1.5 | 3.4 |
| Kinase | | | |
| Bilirubin | 1.3 | 2.6 | 3.4 |
| ALT | 49.1 | 82.8 | 10.3 |

### Activity

Some hints of activity were seen:

2 minor responses in patients with sarcoma and long lasting stabilizations in sarcoma (2) and ovarian cancer (2).

### Conclusions

From the clinical point of view the weekly schedule is really attractive in terms of toxicity. The most frequent toxicities have been reduced more than 3 times. And for instance transaminases toxicity is 8 times less than the 3 hour schedule.

Serious renal toxicity (G3-4) has not been seen with this schedule.

This schedule is very well tolerated and toxicities have been minimized in an profitable way. Though phase I trial are not designed to evaluate efficacy some hints of activity were seen.

1. A method of treating cancer in humans, comprising intravenously infusing a composition comprising ET-743 into a human having cancer at a continuous dosage over a period up to 4 hours, wherein the step of infusing is repeated weekly on a cyclic basis.

2. A method according to claim 1, wherein the cyclic basis comprises one or more weeks of an infusion phase, and one or more weeks of a rest phase, the rest phase not being longer than the infusion phase.

3. A method according to claim 1, wherein the infusion time is from 1 to 3 hours.

4. A method according to claim 1, wherein the infusion time is 2 to 3 hours.

5. A method according to claim 1, wherein the infusion time is about 3 hours.

6. A method according to claim 1, wherein the dosage of Et-743 is below 650 μg/m²/week.

7. A method according to claim 6, wherein the dosage is from 300 to 600 μg/m2/week.

8. A method according to claim 6, wherein the dosage is from 400 to 600 μg/m²/week.

9. A method according to claim 6, wherein the dosage is from 525 to 600 μg/m²/week.

10. A method according to claim 6, wherein the dosage is about 580 μg/m²/week.

11. A method according to claim 1, wherein the cyclic basis comprises weekly administration and a rest phase in each cycle.

12. A method according to claim 11, wherein the rest period is one week within each cycle.

13. A method according to claim 1, wherein each cycle is 2 to 4 weeks.

14. A method according to claim 1, for the treatment of sarcoma, osteosarcoma, ovarian cancer, breast cancer, melanoma, colorectal cancer, mesothelioma, renal cancer, endometrial cancer or lung cancer.

15. A method according to claim 1, wherein another drug is administered to provide a combination therapy.

16. A method according to claim 15, wherein the other drug is selected from:
a) drugs with antimitotic effects, especially those which target cytoskeletal elements, including microtubule modulators such as taxane drugs (such as taxol, paclitaxel, taxotere, docetaxel), podophylotoxins or vinca alkaloids (vincristine, vinblastine);
b) antimetabolite drugs (such as 5-fluorouracil, cytarabine, gemcitabine, purine analogues such as pentostatin, methotrexate);
c) alkylating agents or nitrogen mustards (such as nitrosoureas, cyclophosphamide or ifosphamide);
d) drugs which target DNA such as the antracycline drugs adriamycin, doxorubicin, pharmorubicin or epirubicin;
e) drugs which target topoisomerases such as etoposide;
f) hormones and hormone agonists or antagonists such as estrogens, antiestrogens (tamoxifen and related compounds) and androgens, flutamide, leuprorelin, goserelin, cyprotrone or octreotide;
g) drugs which target signal transduction in tumour cells including antibody derivatives such as herceptin;
h) alkylating drugs such as platinum drugs (cis-platin, carbonplatin, oxaliplatin, paraplatin) or nitrosoureas;
i) drugs potentially affecting metastasis of tumours such as matrix metalloproteinase inhibitors;
j) gene therapy and antisense agents;
k) antibody therapeutics;
l) other bioactive compounds of marine origin, notably the didemnins such as aplidine;
m) steroid analogues, in particular dexamethasone;
n) anti-inflammatory drugs, including nonsteroidal agents (such as acetaminophen or ibuprofen) or steroids and their derivatives in particular dexamethasone; and
o) anti-emetic drugs, including 5HT-3 inhibitors (such as gramisetron or ondasetron), and steroids and their derivatives in particular dexamethasone.

17. A method according to claim 16, wherein the other drug is selected from dexamethasone, doxorubicin, cisplatin, paclitaxel and dexamethasone.

18. The use of ecteinascidin 743 in the preparation of a pharmaceutical composition for a method according to claim 1.

19. A medical kit for administering ET-743, comprising printed instructions for administering ET-743 according to a method of claim 1, and a supply of ET-743 in dosage units for at least one cycle, wherein each dosage unit contains an appropriate amount of ET-743 for the method and a pharmaceutically acceptable carrier.
